(19) United States
(12) Patent Application Publication
(10) Pub. No.: US 2025/0281243 A1
(43) Pub. Date: Sep. 11, 2025

(54) MEDICAL INSTRUMENT FOR USE IN A MAGNETIC RESONANCE TOMOGRAPHY SYSTEM AND METHOD FOR POSITIONING

(71) Applicant: Siemens Healthineers AG, Forchheim (DE)

(72) Inventor: Arne Hengerer, Möhrendorf (DE)

(21) Appl. No.: 19/070,517

(22) Filed: Mar. 4, 2025

(30) Foreign Application Priority Data
Mar. 5, 2024 (DE) 10 2024 202 038.9

(51) Int. Cl. A61B 34/20; G06T 7/00

(52) U.S. Cl. CPC A61B 34/20; G06T 7/00; A61B 2034/2065; G06T 2207/10088

(57) ABSTRACT

A medical instrument for use in a magnetic resonance tomography (MRT) system is provided. The medical instrument has a spirit level for determining a spatial position of the medical instrument. The spirit level contains a liquid that may be detected by the MRT system, so that the location may be detected with the MRT system. A method for alignment of the medical instrument includes determining a trajectory for the medical instrument for a scheduled intervention. An entry point for the medical instrument is identified, and the medical instrument is arranged there. Using a magnetic resonance image of the spirit level, an actual value for a spatial angle of the medical instrument is detected. A target value for the spatial angle is ascertained by the magnetic resonance tomography system, along with information on a deviation that is output to a surgeon. The surgeon corrects the alignment based on the information.

This application claims the benefit of German Patent Application No. DE 10 2024 202 038.9, filed on Mar. 5, 2024, which is hereby incorporated by reference in its entirety.

### BACKGROUND

The present embodiments relate to a medical instrument for use in a magnetic resonance tomography system and to a method for positioning a medical instrument.

Image-based minimally invasive procedures are also performed in magnetic resonance tomography systems. For this, the medical instrument is propagated under real-time control by the magnetic resonance tomography system. To this end, prior to insertion into the abdomen or pelvic region of the patient, the instrument is to be positioned in a predetermined alignment at the insertion point. The success of the intervention or interventions in the course of a working day depends in this case on many factors. For example, the interaction of the surgeon and of the individual components in this case provides a lasting success.

Entry point and entry angle may be planned in the anatomical magnetic resonance image of the patient. The advance of the needle is visualized using real-time magnetic resonance tomography.

The entry angles in two spatial directions are estimated, sometimes by the person performing the intervention placing a finger on the entry point and correcting the alignment of a finger in the MRT image until the finger is approximately in the correct position.

Independent of the grammatical term usage, individuals with male, female, or other gender identities are included within the term.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a medical instrument and a method that simplify and improve alignment are provided.

The medical instrument of the present embodiments is intended for use in a magnetic resonance tomography system (e.g., in a region of the magnetic resonance tomography system that may be detected by magnetic resonance imaging). The term medical instrument refers, for example, also to surgical instruments, such as needles or probes, for example, that are used for biopsy, ablation, or brachytherapy and are intended to be inserted into a patient.

The medical instrument of the present embodiments has a spirit level. The term spirit level in this case refers to all instruments that are configured to use a liquid under the effect of gravity to detect an alignment of the spirit level and thus of a medical instrument connected thereto in a fixed position relative to the force of gravity.

The spirit level is arranged in a predetermined position and location relative to the medical instrument. The spirit level may, for example, be connected to the medical instrument in a fixed position. However, in one embodiment, the alignment of the spirit level may be set to a predetermined value (e.g., with one or more swivel joints), and the alignment may then be fixed in the set location.

For example, a vessel with a concave inner wall that is filled with a liquid and a further fluid of lower density may be provided, so that the fluid collects at the top of the inner wall of the vessel due to buoyancy in a location opposite to the force of gravity. The relative position of the fluid in the vessel in this case indicates an alignment of the vessel to the force of gravity. Thus, for a permanently installed magnetic resonance tomography system with a predetermined position, an alignment of the vessel or spatial location with respect to the magnetic resonance tomography system is also indicated.

The liquid may, in this case, be detected by the magnetic resonance tomography system in a magnetic resonance image. In other words, the location and shape of the liquid may be detected, so that, for example, the shape of the liquid in the vessel and thus also the location of the fluid in the liquid may be detected. The fluid and the liquid have different properties with respect to magnetic resonance tomography imaging, so that the fluid and the liquid may be distinguished in the captured image. The liquid may, for example, be water, and the fluid may, for example, be air or a gas such as oxygen, nitrogen, carbon dioxide, helium, or argon.

The medical instrument of the present embodiments may permit the location of the instrument in the magnetic resonance tomography system to be detected, even without a direct view of the instrument.

The method of the present embodiments is intended for positioning a medical instrument of the present embodiments on a patient using a magnetic resonance tomography system (e.g., using magnetic resonance images).

In one act of the method, a trajectory for the medical instrument is determined for a scheduled intervention. This may take place, for example, by a controller of the magnetic resonance tomography system or a planning system using a magnetic resonance image of the patient and information about the target of the medical instrument in the intervention that is specified by a user or the planning system. The trajectory is in this case a straight line through an entry point on the surface of the patient to the destination, avoiding sensitive organs or structures such as nerves and larger blood vessels.

In a further act, the entry point is identified in accordance with the trajectory relative to the patient, and an alignment of the medical instrument that enables the medical instrument to be moved along the trajectory is ascertained. In this case, identification may be that the entry point of the trajectory that was ascertained on the image or model of the patient is assigned on the real patient and may be marked. Likewise, a spatial direction (e.g., by the controller of the magnetic resonance tomography system) that corresponds to the determined trajectory is determined. This may take place, for example, in that the magnetic resonance tomography system detects a magnetic resonance tomography of the patient, and a location of the patient is detected based on contour and/or organs relative to the magnetic resonance tomography system. From this location, the controller may ascertain a coordinate transformation that transforms the coordinates of the model when determining the trajectory to the real position of the patient in the magnetic resonance tomography system. By applying this coordinate transformation to the determined entry point and the alignment of the medical instrument, the entry point and the alignment on the real patient in the magnetic resonance tomography system are obtained, which may be output by the controller using a visual representation and/or coordinates.

In a further act, the user positions the medical instrument at the entry point on the patient. In one embodiment, in this case, the magnetic resonance tomography system may detect the patient with the medical instrument or a marker on a tip of the instrument facing the patient and may output to the user the position of the determined entry point and the position of the tip (e.g., in a visual representation) as coordinates or a coordinate difference or in the form of an indication of a direction of movement for the instrument to reach the entry point.

In another act, the magnetic resonance tomography system detects at least one actual value of a first spatial angle using a magnetic resonance image of at least one spirit level. A first spatial angle or second spatial angle refers to angles of a polar coordinate system that indicate an alignment in space. The axes of rotation of the spatial angles need not necessarily in this case be perpendicular to one another.

By mapping the liquid and the contrast to the fluid, the location of the fluid becomes apparent. The bubble containing the fluid may be at the highest point of the vessel, opposite to the force of gravity. The external shape of the vessel containing the liquid, reproduced by the fluid, in this case makes the alignment of the vessel relative to the magnetic resonance image apparent. In one embodiment, there may be a mark or scale on the vessel that may be detected with a magnetic resonance acquisition and makes an angle to the perpendicular apparent.

In a further act, the controller determines a target value for the first spatial angle. In other words, the controller ascertains a target value for the first spatial angle, for which, if it is measured by the first spirit level in a corresponding alignment of the medical instrument, the medical instrument is aligned at least in one angular axis in parallel to the predetermined trajectory. In this case, the medical instrument may be rotated about a longitudinal axis, so that the target angle is ascertained by a coordinate transformation from the determined trajectory or its polar coordinates. Depending on the coordinate system selected, the angle that is linearly independent (e.g., that does not depend on another or second spatial angle) may first be selected and set as the first spatial angle.

For example, the controller may perform a coordinate transformation from a coordinate system related to the patient or the magnetic resonance tomography system to a polar coordinate system having reference axes or reference planes that are given by a first spirit level and the second spirit level, or, in the case of a 2D spirit level, by its reference point. The coordinate transformation may, for example, be performed by a matrix multiplication of a vector from the first spatial angle and the second spatial angle using a 2×2 transformation matrix.

The rotation of the medical instrument may, for example, be determined from an alignment of the vessel of the liquid in the magnetic resonance image if the vessel does not have any rotational symmetry about the longitudinal axis of the medical instrument. However, in one embodiment, the vessel of the spirit level may be arranged rotationally symmetrically in the form of a lens and perpendicularly to the longitudinal axis of the medical instrument, as for the two-dimensional spirit level explained below. In this case, the fluid adopts the highest point, and the distance from the center of symmetry is a measure of the inclination of the medical instrument to the perpendicular, given by the force of gravity. The direction of the fluid or of the bubble with respect to a perpendicular straight line through the entry point indicates a second spatial angle.

The controller determines first information as a function of the actual value of the first spatial angle, with which it is possible to achieve the predetermined alignment. This may, for example, be a representation of the target value that indicates to the user together with a representation of the actual value how the user should alter the alignment. In one embodiment, a direction indicator (e.g., an arrow), a sound, or an instruction may be provided to specify to the user the nature of the correction in order to achieve the alignment in accordance with the target value.

In another act, the controller outputs the first information to the user for correction of the alignment of the medical instrument (e.g., on a display or acoustically), so that the user may carry out the correction of the alignment.

The method of the present embodiments may permit a medical instrument of the present embodiments to be aligned precisely in the magnetic resonance tomography system without a direct view thereof.

Further forms of embodiment are specified.

In one possible form of embodiment, the medical instrument has a spirit level that has a first spirit level and a second spirit level. The spirit levels or their vessels in this case may have a longitudinal extension, along which the fluid moves when an inclination of the vessel in a plane in which the longitudinal extension lies is changed. For example, the first spirit level and the second spirit level may take the form of a curved tube.

The second spirit level is arranged at an angle to the first spirit level such that using the first spirit level and the second spirit level an inclination in two linearly independent spatial angles may be detected. In other words, the two spatial angles span a polar coordinate system in space. As a result, an alignment of the medical instrument may be ascertained with the first spirit level and the second spirit level in a polar coordinate system.

Alternatively, however, a two-dimensional spirit level may also be provided as a spirit level, in which the fluid moves along a curved surface (e.g., along the inside of a sphere or a lens that is filled with the liquid). In this case, a location of the vessel in the magnetic resonance image may be detected (e.g., either by a mark, detectable by the magnetic resonance tomography system, on or in the immediate vicinity of the spirit level in a fixed position relative to the spirit level, or by an at least partially asymmetrical shape of the spirit level such as a corner or protrusion or invagination, so that the liquid therein makes it possible to detect the location thanks to the shape in the magnetic resonance image).

A two-dimensional spirit level may simplify the design and also the representation (e.g., if the medical instrument is substantially rotationally symmetrical to its longitudinal axis).

In one possible form of embodiment of the medical instrument, the liquid contains a contrast agent for the magnetic resonance detection. For example, water, Gadolinium, or another substance, for example, may be added to the liquid to accelerate the fall-off of the excitation and thus briefly generate a stronger magnetic resonance signal. The contrast agents are in this case not restricted to those used medically in the body, but because they are enclosed, the spirit level may also, where appropriate, include substances that are incompatible with humans.

The accelerated excitation decay may enable faster rates of image capture for a higher-contrast image.

In one conceivable form of embodiment of the method, the method further has the act of detecting at least one actual value of a second spatial angle using a magnetic resonance image by at least one spirit level. There may in this case also be a second spirit level that, for example, as already explained has an elongated shape and is arranged at an angle to the first spirit level, so that the first spatial angle and the second spatial angle that may be detected by both the spirit levels span a polar coordinate system.

In one embodiment, the first spirit level, as already explained for the two-dimensional spirit level, may be configured to detect a second spatial angle. The two spatial angles may then, for example, be differentiated in a 3D magnetic resonance image by a position of the fluid to a mark on the spirit level or to a predetermined axis or plane in the image. However, two-dimensional slice images or projection images using the magnetic resonance tomography system onto a first plane and a second plane may also be provided. The planes intersect at an angle. The angle may be greater than 5 degrees or 10 degrees. In one embodiment, the angle is 90 degrees. Thanks to the two-dimensional images in intersecting planes, a first spatial angle and a second spatial angle of an alignment of the spirit level and thus of the medical instrument may be determined (e.g., in the 90-degree arrangement of the planes by a simple representation of the images). In one embodiment, a coordinate transformation by the controller may be provided in order to be able to use a preferred coordinate system for a user that, for example, corresponds to the usual coordinate axis designations in magnetic resonance tomography systems.

The detection of the second spatial angle with the magnetic resonance tomography system may enable a full alignment of the medical instrument without visual contact.

In one embodiment, an alignment may be carried out just by measuring an angle (e.g., if the second angle may be detected visually by looking longitudinally into the tunnel). It may then be sufficient if just one spatial angle is detected by the magnetic resonance image, which corresponds to a tilt along the z-axis and cannot be detected visually when looking through the patient tunnel.

In another act, the controller of the magnetic resonance tomography system determines a target value for the second spatial angle. For example, the controller may carry out a coordinate transformation from a coordinate system relating to the patient or the magnetic resonance tomography system to a polar coordinate system having reference axes or reference planes that are given by the first spirit level and the second spirit level, or in the case of a 2D-spirit level, by their reference point. The coordinate transformation may, for example, take place by a matrix multiplication of a vector from the first spatial angle and the second spatial angle using a 2×2 transformation matrix. The target value of the second spatial angle may, in this case, for example, be a function of the first spatial angle (e.g., may be a function of its actual value or its target value).

In a further act, second information on the achievement of the predetermined alignment may be determined as a function of the actual value of the second spatial angle. In most cases, this is also a function of the actual value of the first spatial angle. In other respects, what is said above about the first information applies (e.g., as regards the type of information).

In another act, the second information is output to the user for correction of the alignment of the medical instrument (e.g., for correction of the actual value for the second spatial angle).

The method may also allow a correction in two spatial angles and thus a full alignment in parallel to the trajectory

In a form of embodiment of the method, the user then carries out a correction of the alignment of the medical instrument in accordance with the first information and/or second information. In other words, based on the information, the user moves the medical instrument such that a deviation of the actual value from the target value of the first spatial angle and/or second spatial angle becomes smaller. The information may in this case specify the direction, or the information represents target and actual values for the user, so that the user may intuitively infer the direction for correction (e.g., in the iterative method described below).

In a form of embodiment of the method, the acts of detecting an actual value, determining information, outputting the information, and correcting the alignment are repeated for the first spatial angle and/or the second spatial angle.

The user may thus track the success of his or her correction, and for example, when the angular corrections are a function of one another, the user is thus guided more reliably and faster to the desired result.

In one possible form of embodiment, the controller of the magnetic resonance tomography system determines a deviation between target value and actual value (e.g., via a sum of squares of the differences). The deviation is compared by the controller with a predetermined threshold value and signaled to the user by a message if the value falls below the threshold value. This may take place, for example, by an acoustic signal or information on a display. In one embodiment, the controller may automatically abort a repetition of the previously explained acts because the target has been reached thanks to the iteration. However, it is also possible for this to take place only following an input from the user for confirmation.

In the method, the user may be notified if the alignment is achieved with sufficient accuracy and the intervention may be started.

### BRIEF DESCRIPTION OF THE DRAWINGS

The above-described properties, features, and advantages of the present embodiments and the manner in which they are achieved will become clearer and more readily understandable in connection with the following description of the example embodiments, which are explained in greater detail in connection with the drawings, in which:

FIG. 1 shows a schematic representation of a form of embodiment of a magnetic resonance tomography system for the execution of a method of the present embodiments;

FIG. 2 shows a form of embodiment of a medical instrument;

FIG. 3 shows a further form of embodiment of a medical instrument; and

FIG. 4 shows a schematic flow chart of an embodiment of a method.

### DETAILED DESCRIPTION

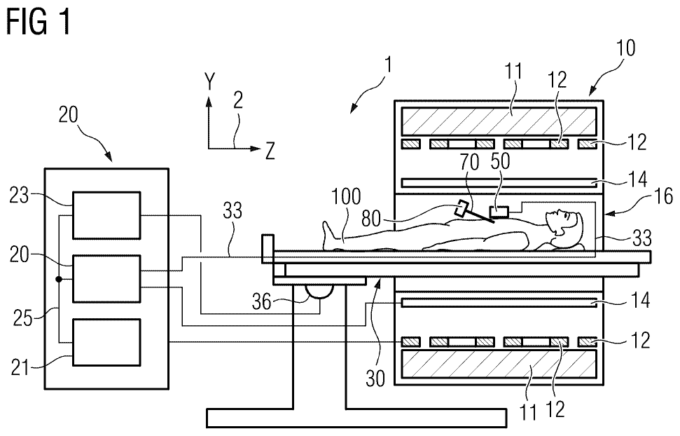

FIG. 1 shows a schematic representation of a form of embodiment of a magnetic resonance tomography system 1 for execution of an embodiment of a method with an embodiment of a medical instrument 70.

A magnet unit 10 has a field magnet 11 that generates a static magnetic field B0 for alignment of nuclear spins of samples or of a patient 100 in an acquisition region. The acquisition region is characterized by an extremely homogeneous static magnetic field B0, where the homogeneity relates, for example, to a magnetic field strength or an absolute value. The acquisition region is virtually spherical and is arranged in a patient tunnel 16 that extends in a longitudinal direction 2 through the magnet unit 10.

A patient couch 30 may be moved in the patient tunnel 16 by a displacement unit 36.

The field magnet 11 may be a superconducting magnet that may provide magnetic fields with a magnetic flux density of up to 3T, and even higher in the latest devices. However, for lower field strengths, permanent magnets or electromagnets with normally conducting coils may also be used.

The magnet unit 10 further has gradient coils 12 that are configured to superimpose variable magnetic fields in three spatial directions on the magnetic field B0, for spatial differentiation of detected imaging areas in an examination volume. The gradient coils 12 may be coils made of normally conducting wires that may generate fields orthogonal to one another in the examination volume.

The magnet unit 10 likewise has a body coil 14 that is configured to emit a radio-frequency signal supplied via a signal line into the examination volume, receive resonance signals emitted by the patient 100, and emit the received resonance signals via a signal line. In the following, the term transmitting antenna refers to an antenna via which the radio-frequency signal is emitted to excite the nuclear spins. This may be the body coil 14, but also a local coil 50 with a transmitting function.

A control unit 20 supplies the magnet unit 10 with the various signals for the gradient coils 12 and the body coil 14 and evaluates the received signals.

Thus, the control unit 20 has a gradient controller 21 that is configured to supply the gradient coils 12 via supply lines with variable currents that provide the desired gradient fields in the examination volume in a time-coordinated manner.

The control unit 20 further has a radio-frequency unit 22 that is configured to generate a radio-frequency pulse with a specified timing, amplitude, and spectral power distribution for the excitation of a magnetic resonance of the nuclear spins in the patient 100. In this case, pulse powers in the kilowatt range may be achieved. The excitation signals may be radiated into the patient 100 via the body coil 14 or else via a local transmitting antenna.

A controller 23 communicates with the gradient controller 21 and the radio-frequency unit 22 via a signal bus 25.

Arranged on the patient 100 is a local coil 50 that is connected to the radio-frequency unit 22 and its receiver via a connection line 33. However, in one embodiment, the body coil 14 may be a receiving antenna within the present embodiments.

In accordance with the application, the medical instrument 70 is arranged by a surgeon on the patient 100 in the patient tunnel 16 in the detection area of the magnetic resonance tomography system. The medical instrument 70 may, for example, be a biopsy needle, an instrument for a brachytherapy, or another instrument that is to be inserted into the body of the patient in connection with an interventional procedure. For this, the medical instrument is placed at a predetermined entry point on the surface or skin of the patient and is aligned along a trajectory to a target location in the body of the patient in accordance with a predetermined inclination.

Arranged on the medical instrument 70 is a spirit level 80 that is described in greater detail in the following figures.

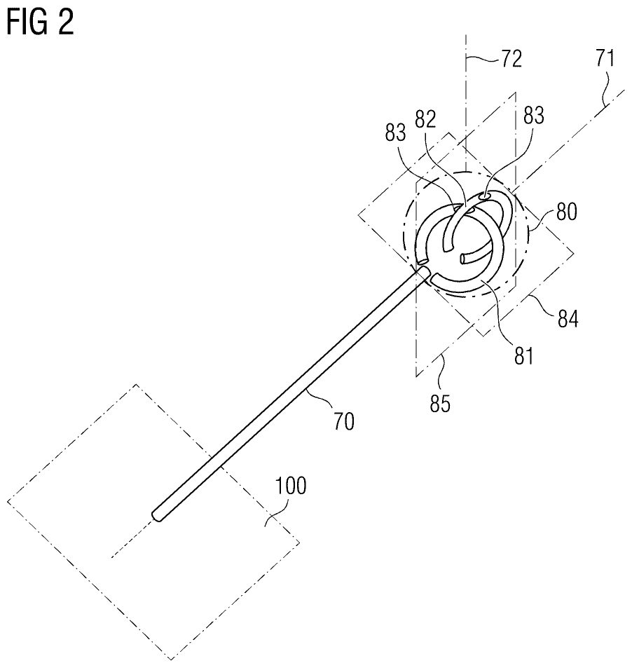

FIG. 2 shows a form of embodiment of a medical instrument 70. The medical instrument 70 has a longitudinal extension 71 at a distal end, which in a position in accordance with the application faces away from the patient 100, a spirit level 80. The spirit level 80 has a first spirit level 81 and a second spirit level 82. The first spirit level 81 is arranged in a first plane 84, and the second spirit level 82 is arranged in a second plane 85. Both planes 84, 85 may be aligned in parallel to the longitudinal axis 71 of the medical instrument 70 and confine an angle that may correspond to 90 degrees. However, in one embodiment, the planes 84, 85 may be aligned differently, providing the planes 84, 85 are not parallel to one another. However, additional coordinate transformations are then required in the subsequent method when aligning the medical instrument 70.

The first spirit level 81 may, for example, have a curved tube that extends in the first plane 84 in at least a partial circle. The tube is filled with a liquid that may be detected by the magnetic resonance tomography system in a magnetic resonance image, except for a vial or bubble consisting of a fluid with a lower density compared to the liquid. Under the effect of gravity on the liquid, the vial always positions itself at the highest point of the tube in the opposite direction to the force of gravity or to the vertical 72. In conjunction with the position of the tube, which is reflected by the liquid or other markers visible in a magnetic resonance image having a predetermined position relative to the tube, an alignment angle of the tube, and thus of the first spirit level 81 and the medical instrument associated therewith, may be ascertained in the first plane 84 to the vertical 72.

The second spirit level 82 may be substantially identical to the first spirit level 81, but is merely arranged in the second plane 85, which is arranged at an angle to the first plane (e.g., greater than 10 degrees, 30 degrees, or 45 degrees or equal to 90 degrees), so that the angles to the vertical detected by both the spirit levels 81, 82 in both planes 84, 85 in each case span a polar coordinate system. With a knowledge of the location of the planes, an alignment of the medical instrument may be determined.

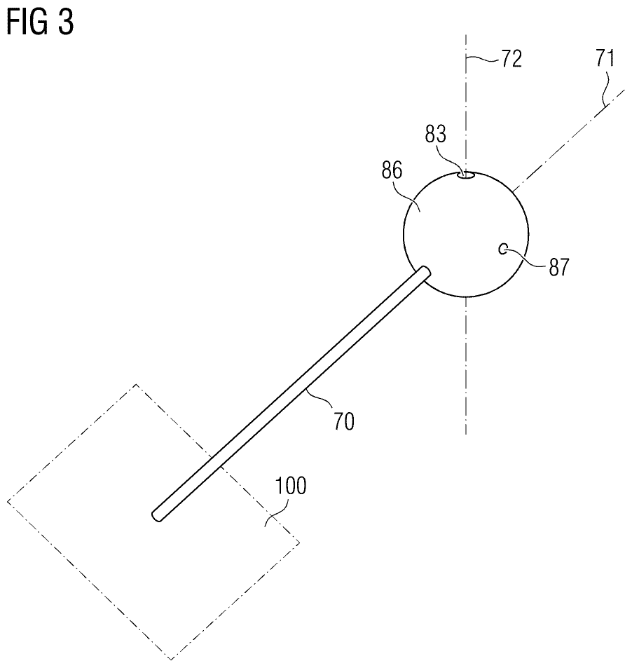

FIG. 3 shows another form of embodiment of the medical instrument 70, which differs by the type of spirit level 80. Whereas in FIG. 2 a first spirit level 81 and a second spirit level 82 detect two spatial angles separately in the first plane 84 and in the second plane 85, the spirit level 80 in FIG. 3 is a two-dimensional spirit level 86, also referred to as a circular bubble. However, the two-dimensional spirit level 86 is not embodied as a flat lens, but has a greater height (e.g., as a hemisphere or sphere). As a result, the two-dimensional spirit level is able, in contrast to the circular bubble, to detect larger angles and thus larger angular deviations in the alignment of the longitudinal axis 71 to the vertical 72.

A mark 87 may be arranged on the spirit level 80 or the medical instrument 70, and may be detected in an image using the magnetic resonance tomography system 1. Thus, an orientation of the medical instrument about the longitudinal axis 71 may be detected, which due to the rotational symmetry of the spirit level 80, cannot be detected via the liquid.

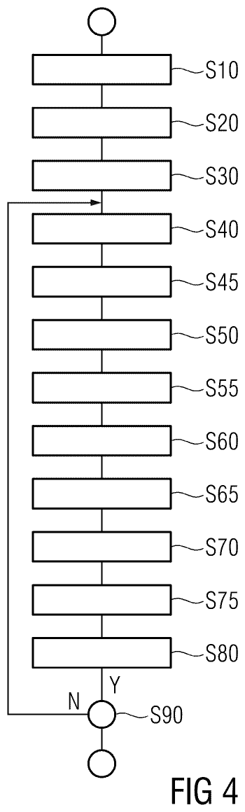

FIG. 4 shows a schematic flow chart of a form of embodiment of the method for positioning a medical instrument using a magnetic resonance tomography system.

First, in act S10, a trajectory for the medical instrument is determined for a scheduled intervention. This may take place with the aid of a planning program that, on a controller 23 of the magnetic resonance tomography system 1, uses a magnetic resonance image of the patient 100 to determine a path of the medical instrument 70 from an entry point on the patient 100 to a target area on or in an organ, this being referred to as a trajectory.

In a further act S20, an entry point on the patient 100 and an alignment of the medical instrument 70 is determined, enabling the medical instrument to be moved along the trajectory. The alignment may be defined based on two angles that may be detected by the spirit level 80 and a magnetic resonance image. However, only one angle that, for example, extends with the plane in which it lies in the longitudinal direction or z-axis may be used, since this angle is difficult for a surgeon to detect visually. The other angle (e.g., in a plane perpendicular to the z-axis) is then detected visually by the surgeon.

In the form of embodiment in FIG. 2, two angular values to be set for the first spirit level 81 and the second spirit level 82 may be determined by coordinate transformation from a coordinate system related to the magnetic resonance tomography system 1 or the patient 100 (e.g., with the angles of inclination to the vertical 72 and an angle of rotation about the vertical 72 in relation to the z-axis 2 or longitudinal extension of the patient tunnel). In this case, a predetermined alignment of the first plane 84 with the first spirit level 81 and of the second plane 85 with the second spirit level 82 may be adopted (e.g., the first plane 84 in parallel to the z-axis and the second plane 85 perpendicular to the z-axis), which in the subsequent act S30, the surgeon has to adopt with the medical instrument 70. Or in act S30, the alignment of the first spirit level 81 and of the second spirit level 82 is detected using a magnetic resonance image; only then in act S20 are both the target angles for the first spirit level 81 and the second spirit level 82 determined.

In contrast, in the form of embodiment in FIG. 3, due to the rotational symmetry of the two-dimensional spirit level 86, only the inclination compared to the vertical 72 may be detected via the position in the vial 83 with respect to the two-dimensional spirit level 86. In contrast, the position of the vial 83 relative to the entry point or the patient 100 or also to a mark 87 on the medical instrument 70 (e.g., at the tip at the entry point) that may be detected with the magnetic resonance image provides a second angular coordinate.

In a further act S30, the user or the surgeon positions the medical instrument 70 at the entry point on the patient 100. For this, the user moves the tip of the medical instrument 70 to the entry point, which, for example, in a preceding step, was marked using real-time mapping with the magnetic resonance tomography system 1 with the finger or a magnetic-resonance-active substance.

In another act S40, an actual value of a first spatial angle is detected. For this, a magnetic resonance image of the spirit level 80 or of the first spirit level 81 and/or of the second spirit level 82 or of the two-dimensional spirit level 86 is detected. The fluid of the vial 83 contrasts with the liquid in the spirit level 80 and thus indicates at least an angle of inclination by its position in the spirit level 80.

In a further act S50, the controller 23 of the magnetic resonance tomography system 1 determines a target value for the first spatial angle. In this case, the target value for the first spatial angle may be a function of the actual value if the coordinate system of the medical instrument 70 is tilted compared to the coordinate system of the magnetic resonance tomography system 1. The target value may, for example, be determined by a coordinate transformation of the alignment of the trajectory.

In another act S60, the controller 23 determines first information for the achievement of the predetermined alignment as a function of the actual value of the first spatial angle. For example, on a display, a directional arrow may indicate a direction in which the medical instrument is to be inclined, or in which the inclination is to be altered in order to achieve or come closer to the target value.

In a further act S70, the controller outputs the first information to the user. This may be an output or representation on a display, graphically or as an instruction. Also possible is an acoustic output by a sound, a sound sequence, or as voice output.

Then, based on the information output, the user may correct the alignment of the medical instrument 70 or approximate the target value.

In one possible form of embodiment of the method, the method may further have the act S45 of detecting an actual value of a second spatial angle using a magnetic resonance image of at least one spirit level. In the case of the two-dimensional spirit level 80, this may be an angle in a second plane of intersection, or in the case of the spirit level in FIG. 2, a displacement angle of the second spirit level 82 in the second plane 85.

In act S55, the controller 23 finally determines a target value for the second spatial angle. The target value for the second angle may in this case be a function of the actual value of the first spatial angle and the determination accordingly a function of the actual value(s) of the first spatial angle and/or of the second spatial angle.

In act S65, the controller determines, in accordance with act S60, second information for the achievement of the predetermined alignment as a function of the actual value of the second spatial angle and/or first spatial angle, and in act S75, outputs this information to the user for correction of the alignment of the medical instrument, as set out for act S70.

The alignment may take place iteratively in one form of embodiment of the method. This provides that the acts of capturing an actual value S40, S45, determining a target value S50, S55, determining information S60, S65, outputting the information S70, and correcting the alignment are repeated for the first spatial angle and/or second spatial angle until the medical instrument 70 has achieved the determined alignment along the trajectory. For this, the controller 23 of the magnetic resonance tomography system 1 may determine, in act S90, a deviation between target value and actual value and may signal to the user if the deviation between the alignment of the longitudinal axis 71 of the medical instrument 70 and the determined trajectory falls below a predetermined threshold value. The deviation may in this case, for example, be measured by the sum of squares of the differences for actual value and target value of the first spatial angle and of the second spatial angle.

Although the invention has been illustrated and described in greater detail by the example embodiments, the invention is not restricted by the disclosed examples, and other variations can be derived therefrom by the person skilled in the art, without departing from the scope of protection of the invention.

The elements and features recited in the appended claims may be combined in different ways to produce new claims that likewise fall within the scope of the present embodiments. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A medical instrument for use in a magnetic resonance tomography system, the medical instrument comprising:
a spirit level for determining a spatial location of the medical instrument,
wherein the spirit level is arranged in a predetermined position and location relative to the medical instrument and contains a liquid that is detectable by the magnetic resonance tomography system.

2. The medical instrument of claim 1, wherein the spirit level has a first spirit level and a second spirit level that is arranged at an angle to the first spirit level, such that using the first spirit level, a first inclination is detected, using the second spirit level, a second inclination is detected, and a spatial angle of the first inclination and a spatial angle of the second inclination span a polar coordinate system in space.

3. The medical instrument of claim 1, wherein the liquid contains a contrast agent for magnetic resonance detection.

4. A method for positioning a medical instrument on a patient using a magnetic resonance tomography system, the medical instrument comprising a spirit level for determining a spatial location of the medical instrument, wherein the spirit level is arranged in a predetermined position and location relative to the medical instrument and contains a liquid that is detectable by the magnetic resonance tomography system, the method comprising:
determining a trajectory for the medical instrument for a scheduled intervention;
identifying an entry point on the patient and an alignment of the medical instrument that enable the medical instrument to move along the trajectory;
positioning the medical instrument at the entry point by a user;
detecting at least one actual value of a first spatial angle using a magnetic resonance image of the spirit level;
determining a target value for the first spatial angle by a controller of the magnetic resonance tomography system;
determining first information for achievement of a predetermined alignment as a function of the at least one actual value of the first spatial angle; and
outputting the first information to the user for correction of the alignment of the medical instrument.

5. The method of claim 4, further comprising:
detecting at least one actual value of a second spatial angle using a magnetic resonance image of the spirit level;
determining a target value for the second spatial angle by a controller of the magnetic resonance tomography system;
determining second information for achievement of the predetermined alignment as a function of the at least one actual value of the second spatial angle; and
outputting the second information to the user for correction of the alignment of the medical instrument.

6. The method of claim 4, further comprising correcting, by the user, an alignment of the medical instrument in accordance with the first information, the second information, or the first information and the second information.

7. The method of claim 6, wherein detecting an actual value of the at least one actual value, determining the target value, determining the first information, outputting the first information, and correcting the alignment are repeated for the first spatial angle, the second spatial angle, or the first spatial angle and the second spatial angle.

8. The method of claim 7, further comprising determining, by the controller of the magnetic resonance tomography system, a deviation between the target value and the actual value and signals to the user when the deviation falls below a predetermined threshold value.

9. A magnetic resonance tomography system for positioning a medical instrument, the medical instrument comprising a spirit level for determining a spatial location of the medical instrument, wherein the spirit level is arranged in a predetermined position and location relative to the medical instrument and contains a liquid that is detectable by the magnetic resonance tomography system, the magnetic resonance tomography system comprising:
a controller configured to:
detect at least one actual value of a first spatial angle of the medical instrument positioned at an entry point on a patient using a magnetic resonance image of the spirit level;
capture a target value for the first spatial angle;
determine information for achievement of a predetermined alignment as a function of the at least one actual value of the first spatial angle; and
output the information to the user for correction of an alignment of the medical instrument.
